(19) United States
(12) Patent Application Publication
(10) Pub. No.: US 2026/0137322 A1
(43) Pub. Date: May 21, 2026

(54) POWER AND GROUND PATH ISOLATION CIRCUITRY

(71) Applicant: Meta Platforms Technologies, LLC, Menlo Park, CA (US)

(72) Inventors: Robert Michael Proie, JR., Aldie, VA (US); Raffael Engleitner, Morris Township, NJ (US); Aziz Yuldashev, Brooklyn, NY (US)

(21) Appl. No.: 18/060,123

(22) Filed: Nov. 30, 2022

Related U.S. Application Data
(60) Provisional application No. 63/369,162, filed on Jul. 22, 2022.

(51) Int. Cl. A61B 5/296; A61B 5/00

(52) U.S. Cl. CPC A61B 5/296; A61B 5/0004

(57) ABSTRACT

Systems, devices, and methods for power and ground path isolation are provided. In some embodiments, a wearable device comprises: a plurality of electrodes, each electrode configured to sense physiological signals of a wearer of the wearable device; a charging circuit configured to operatively couple to an external charger during charging of the wearable device; a set of charging contact interfaces comprising a charging contact and a ground contact; and an isolation circuit configured to operatively couple to the set of charging contact interfaces and the charging circuit. The isolation circuit may comprise: a first transistor configured to block leakage current via the charging contact while in an off state of the first transistor; and a second transistor configured to block leakage current via the ground contact while in an off state of the second transistor.

### RELATED APPLICATIONS

This application claims the benefit of U.S. Provisional Application No. 63/369,162, filed Jul. 22, 2022, entitled “POWER AND GROUND PATH ISOLATION CIRCUITRY”, which is assigned to the assignee hereof, and incorporated herein in its entirety by reference.

### BACKGROUND

Measuring signals via electrodes on body-worn devices may be challenging for various reasons. For example, parasitic leakage paths may cause measuring signals via such electrodes to be difficult. However, addressing such parasitic leakage paths may be difficult, particularly for portable devices.

### SUMMARY

Systems, devices, and methods for power and ground path isolation are provided. According to some embodiments, a wearable device may comprise: a plurality of electrodes, each electrode configured to sense physiological signals of a wearer of the wearable device; a charging circuit configured to operatively couple to an external charger during charging of the wearable device; a set of charging contact interfaces comprising a charging contact and a ground contact, wherein the set of charging contact interfaces are operatively coupled to the charging circuit; and an isolation circuit configured to operatively couple to the set of charging contact interfaces and the charging circuit, wherein the isolation circuit comprises. In some embodiments, the isolation circuit comprises: a first transistor configured to block leakage current via the charging contact while in an off state of the first transistor, the off state of the first transistor corresponding to a time during which charging of the wearable device is not occurring; and a second transistor configured to block leakage current via the ground contact while in an off state of the second transistor, the off state of the second transistor corresponding to a time during which charging of the wearable device is not occurring.

In some examples, the wearable device further comprises a third transistor operatively coupled to a source terminal of the first transistor, wherein the third transistor is configured to switch the first transistor from the off state to an on state. In some examples, the switch of the first transistor from the off state to the on state is further controlled by parameters of a second resistor and a capacitor in series with the third transistor. In some examples, a distortion of a pulse applied at the charging contacts is used to detect foreign substances on the charging contacts, wherein the distortion is due to the second resistor and the capacitor.

In some examples, the wearable device further comprises a resistor operatively coupled to the charging contact and the ground contact, and operatively coupled to a gate terminal of the first transistor and to a source terminal of the second transistor. In some examples, the resistor causes a difference in a voltage at the charging contact and the ground contact to be about 0 V. In some examples, the resistance of the resistor is usable to detect a presence of the isolation circuit.

In some examples, the physiological signals are electromyography (EMG) signals.

In some examples, the wearable device is a wrist-worn device. In some examples, the plurality of electrodes are disposed around a wrist band of the wrist-worn device.

In some examples, the wearable device further comprises a bias voltage electrode operatively coupled to bias voltage circuitry, wherein the bias voltage circuitry is configured to apply a bias voltage to skin of the wearer via the bias voltage electrode.

According to some embodiments, a method for power and ground path isolation comprises: responsive to a wearable device being in a non-charging state, causing leakage current via a charging contact of the wearable device to be blocked and causing leakage current via a ground contact of the wearable device to be blocked, wherein the charging contact and the ground contact are disposed on a portion of the wearable device configured to be in contact with skin of the wearer of the wearable device; and responsive to the wearable device being in a charging state, causing electrical energy from an external charger operatively coupled to the wearable device to be stored in a battery of the wearable device.

In some examples, causing leakage current via the charging contact to be blocked comprises causing a first transistor to be in an off state. In some examples, causing leakage current via the ground contact to be blocked comprises causing a second transistor to be in an off state.

In some examples, during at least a portion of the charging state, a first transistor associated with the charging contact and a second transistor associated with the ground contact are both in an on state. In some examples, the first transistor and the second transistor are transitioned to the on state by causing a third transistor to be in an on state. In some examples, the third transistor is transitioned to the on state responsive to the external charger being operatively coupled to the wearable device, and wherein the third transistor transitions to the on state with a delay after the external charger is operatively coupled to the wearable device.

In some examples, the wearable device comprises a plurality of electrodes configured to sense physiological signals of the wearer. In some examples, the plurality of electrodes comprise a plurality of electromyography (EMG) electrodes.

In some examples, the wearable device further comprises a bias electrode configured to apply a bias voltage to the skin of the wearer.

### BRIEF DESCRIPTION OF DRAWINGS

Illustrative embodiments are described in detail below with reference to the following figures.

FIG. 1 is a plan view of an example wristband system in accordance with some embodiments.

FIG. 2 is a schematic diagram of an example device that includes circuitry for power and ground path isolation in accordance with some embodiments.

FIG. 3 is a schematic diagram of a circuit for power and ground path isolation in accordance with some embodiments.

FIG. 4 is a flowchart of an example process for power and ground path isolation in accordance with some embodiments.

### DETAILED DESCRIPTION

Measuring signals via electrodes, particularly dry electrodes, may be challenging. For example, due to high impedances between dry electrodes and a wearer's skin, there may be parasitic leakage paths between an electrode and the skin. Additionally, for devices that are portable, such as wearable user devices (e.g., smart watches, arm bands, chest bands, rings, etc.), to reduce power consumption and enable more robust sensing, a bias voltage may be applied to the body, e.g., via a bias electrode. Parasitic leakage paths may cause the voltage between the wearer's body and the system ground to drift away from the applied bias voltage. This may in turn limit analog front end (AFE) circuit dynamic range, which may in turn affect the quality of signals sensed by the electrodes. An example of electrodes that may be disposed on a wearable device are electrodes used for sensing electromyography (EMG) signals indicative of neuromuscular signals used to activate and/or more underlying muscles of the wearer.

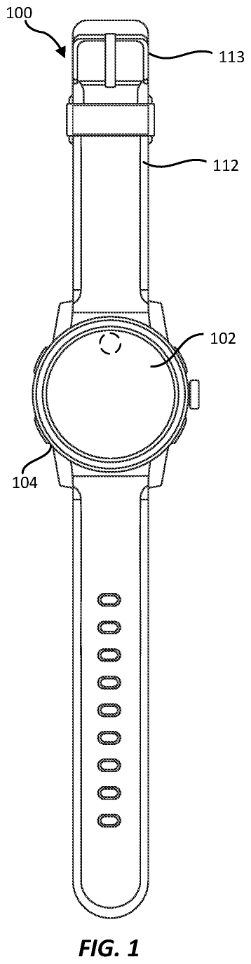

An example of a wearable user device that may include sensing electrodes is a wrist-worn device, such as a smart watch or fitness tracker. FIG. 1 illustrates an example wristband system 100 that includes a watch body 104 coupled to a watch band 112. Watch body 104 and watch band 112 may have any size and/or shape that is configured to allow a user to wear wristband system 100 on a body part (e.g., a wrist). Wristband system 100 may include a retaining mechanism 113 (e.g., a buckle) for securing watch band 112 to the user's wrist. Information, such as the time, date, measured user characteristics (e.g., physiological characteristics), etc. may be displayed on display 102. Display 102 may be a touchscreen such that the user may navigate through, e.g., menus, by touching portions of display 102. Wristband system 100 may perform various functions associated with the user. The functions may be executed independently in watch body 104, independently in watch band 112, and/or in communication between watch body 104 and watch band 112. Watch band 112 may be configured to operate independently (e.g., execute functions independently) from watch body 104. Additionally or alternatively, watch body 104 may be configured to operate independently (e.g., execute functions independently) from watch band 112. In some implementations, watch band 112 and/or watch body 104 may each include the independent resources required to independently execute functions. For example, watch band 112 and/or watch body 104 may each include a power source (e.g., a battery), a memory, data storage, a processor (e.g., a CPU), communications, a light source (e.g., at least one infrared LED for tracking watch body 104 and/or watch band 112 in space with an external sensor), and/or input/output devices. In some implementations, EMG electrodes for sensing EMG signals may be disposed in and/or on a portion of wristband system 100 that is configured to be in contact with the user's skin. For example, electrodes may be disposed on a back portion of watch band 112, a back portion of watch body 104, or any combination thereof.

For wearable user devices, the battery charging interface may present a parasitic leakage path. For example, a wearable user device, such as a smart watch, may have charging contacts on the underside of the device. As a more particular example, charging contacts of a smart watch may be on the underside of a capsule of the watch. When not being charged, these charging contacts may come in contact with the user's skin, either due to direct physical contact, or due to a foreign body (e.g., sweat), bridging the space between the skin and the charging contacts.

Conventional techniques for isolation circuitry have various limitations. For example, such isolation circuitry may require an internal voltage (e.g., from the device battery) to be present and may therefore not be operational when the device battery is dead or nearly dead. As another example, such isolation circuitry may not block currents by default, or may only block current above some threshold amount (e.g., more than 10 seconds of milliamp levels of leakage current), which is not sufficient for many applications, such as EMG sensing with dry electrodes. Moreover, such isolation circuitry may not provide timing control over when leakage current is blocked, and/or may be exclusive to isolating either a high path or a low path. More robust isolation circuitry may require more space (e.g., may have a higher form factor), which is undesirable in wearable devices.

Disclosed herein is circuitry configured to reduce leakage associated with a charging path for a wearable device. The circuitry may be configured to block leakage current during signal sensing (e.g., via one or more EMG electrodes) in order to allow for robust signal acquisition. Moreover, the circuitry may be configured to allow device charging without firmware control, e.g., in the case of a completely dead battery. The circuitry disclosed herein may be configured to detect foreign substances (e.g., water, sweat, etc.). The circuitry disclosed herein may have a relatively small form factor such that the circuitry can be disposed within electronics packaging of a wearable user device, such as a smart watch.

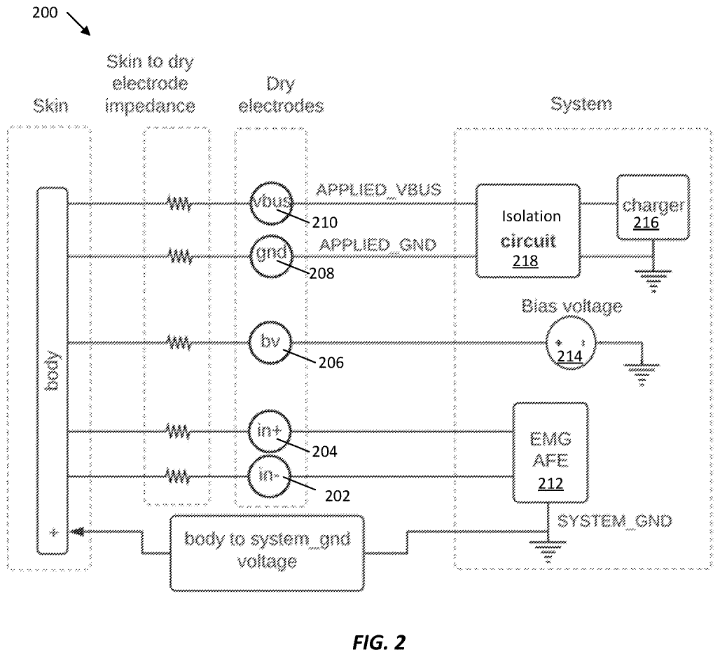

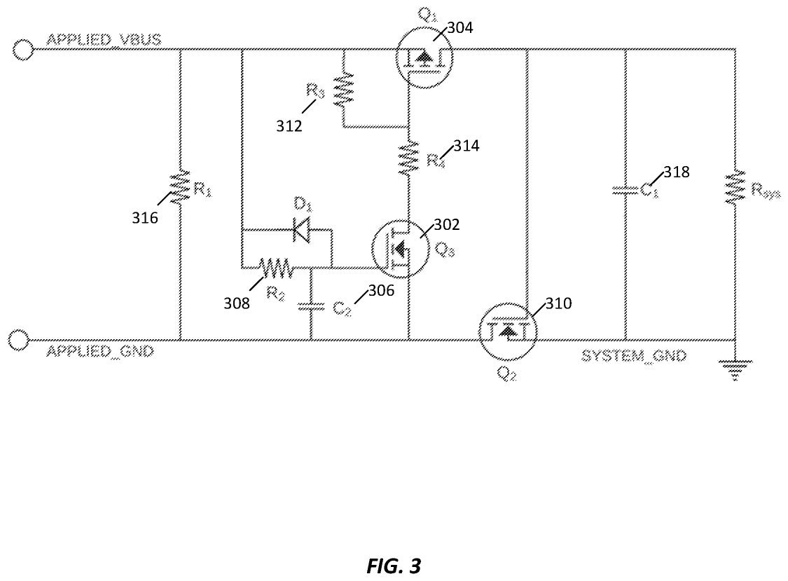

FIG. 2 is a schematic diagram of an example wearable device 200 that includes an example power and ground path isolation circuit in accordance with some embodiments. As illustrated, wearable device 200 includes a set of electrodes, at least a subset of which may be configured to be in contact with the body or skin of the user. The electrodes may include EMG electrodes (e.g., EMG electrodes 202 and 204), a bias voltage electrode 206, and one or more charging contacts (e.g., charging contact 210 and ground contact 208). Signals from EMG electrodes 202 and 204 may be provided to EMG analog front end (AFE) 212, as shown in FIG. 2. The bias voltage applied to bias voltage electrode 206 may be applied and/or generated via bias circuitry 214. Examples of bias voltages that may be applied are 2V, 2.5V, 3V, 3.5V, etc. Charging voltage (e.g., as supplied by an external charger) may be provided to charging circuitry 216. Charging circuitry 216 may serve to, e.g., store energy in a battery of wearable device 200, monitor a current available capacity of the battery, or the like. In some embodiments, an isolation circuit 218 may be operatively coupled between the charging contacts (e.g., charging contacts 208 and 210) and charging circuitry 216. An example of isolation circuit 218 is shown in and described below in connection with FIG. 3.

FIG. 3 is a schematic diagram of an example power and ground path isolation circuit in accordance with some embodiments. For example, isolation circuit 218 of FIG. 2 may be similar to what is shown in FIG. 3. At times at which the wearable device is not being charged (generally referred to herein as “a non-charging state”), resistor R1 ensures that the potential difference between the APPLIED_VBUS and APPLIED_GND is 0 Volts, regardless of whether one or more charging contacts come into electrical contact with the skin (e.g., whether by coming into direct physical contact, or via a foreign body such as a sweat). By keeping the potential difference between APPLIED_VBUS and APPLIED_VGND low (e.g., substantially near 0 V), both transistor 304 (Q1) and transistor 310 (Q2) may remain in an OFF state. Maintaining transistor 304 (Q1) and transistor 310 (Q2) in an OFF state may prevent current from either branch from flowing to the system ground (e.g., SYSTEM_GND, as indicated in FIG. 3). Note that in the example power and ground path isolation circuit shown in FIG. 2, due to transistors 304 (Q1) and 310 (Q2) being in an OFF state, leakage current is blocked on both the high path (via transistor 304 being in the OFF state) and the low path (via transistor 310 being in the OFF state). In some implementations, transistor 310 may be replaced by a diode. In some implementations, transistors 302, 304, and/or 310 may be selected based on a voltage drop across each transistor, e.g., to limit the voltage drop to less than a predetermined threshold (e.g., less than a tenth of the voltage headroom of the circuit, or the like). Note that during the non-charging state, EMG signals may be sensed, e.g., as the wearable device is worn by the user. Additionally, note that the non-charging state may include both times at which an external charger is not operatively coupled to the charging contacts, as well as times during which the external charger is operatively coupled to the charging contacts but due to impedance measurement by a processor associated with the external charger, charging is inhibited (e.g., due to detection of liquid or dirt at the charging contacts).

During a “charging state,” which is generally referred to as a time in which an external charger is operatively coupled to the charging contacts and during which the external charger is providing a charging voltage and/or current to charge a battery of the wearable device, transistors 302 (Q3), 304 (Q1), and 310 (Q2) may transition to an ON state. In particular, upon a charging voltage being provided to isolation circuit 200, transistor 302 (Q3) may turn on with a time delay dictated by the values of resistor 308 (R2) and capacitor 306 (C2). Transistor 302 (Q3) transitioning to the ON state may cause transistor 304 (Q1) and transistor 310 (Q2) to each transition to the ON state. The time delay associated with transistor 302 (Q3) transitioning to the ON state may be used to detect the presence of a foreign object (e.g., sweat, liquid, etc.) on the charging contacts, as described below.

During charging, a processor of an external charger may be configured to detect the presence of the power and ground path isolation circuit. For example, the external charger may be configured to detect the presence of the isolation circuit by measuring an impedance associated with isolation circuit 200. Responsive to determining that the measured impedance corresponds to the resistance of resistor 316 (R1), the processor of the external charger may detect that the presence of the isolation circuit, verify that the correct wearable device is connected to the external charger (e.g., that the wearable device corresponds to the external charger), or the like. The processor of the external charger may additionally be configured to measure the impedance (which may include resistance and capacitance) of the interface to ensure that no foreign substances (e.g., water, sweat, etc.) are present. In particular, the processor of the external charger may apply a pulse (e.g., a voltage pulse) and determine a distortion in the pulse based on the time constant of resistor 308 (R2) and capacitor 306 (C2). The processor of the external charger may then responsively inhibit charging, cause an alert to be presented (e.g., on a display of the wearable device), etc., to protect the wearable device. Note that measurement of the impedance may occur during a time window that spans a first time point when the external charger is connected to the wearable device and a second time point at which transistor 302 (Q3) transitions to an ON-state. This time window is controlled by the values of resistor 308 (R2) and capacitor 306 (C2). During this time window, the system capacitance 318 (C1) may be masked in order to improve the signal-to-noise ratio (SNR) of the impedance measurement. Note that capacitor 318 (C1) may be generally in the microfarad range and may serve to ensure stable operation of the internal circuitry. Because capacitor 318 (C1) may have a fixed value (e.g., cannot be reduced), masking capacitor 318 (C1) may be performed in order to measure the impedance, and accordingly, detect foreign substances.

The power and ground path isolation circuit shown in and described above in connection with FIG. 3 may have various benefits and/or characteristics. For example, the parasitic leakage current may be controlled (e.g., during measurement of EMG signals) to nAmp levels. The leakage current may be dictated by the drain-source leakage current (e.g., I_{DSS}) and gate-source leakage current (e.g., I_{GSS}) of transistors 304 (Q1) and 310 (Q2). As another example, an equal potential may be maintained between the charging and ground charging contacts via resistor R1. As yet another example, the isolation circuit may prevent or block current flow from external signals to internal power and ground paths when the input voltage is below a predefined level or sustained below a predefined time via transistors 304 (Q1) and 310 (Q2). As still another example, the isolation circuit may allow for the internal capacitance 318 (C1) to be masked from external systems to allow for foreign material assessment (e.g., water or sweat detection) for a predefined time. This may be controlled by controlling (e.g., delaying) a turn-on time of transistor 304 (Q1) via transistor 302 (Q3), resistor 308 (R2), and capacitor 306 (C2). The isolation circuit may be configured to operate with a fully or nearly dead battery without relying on firmware to control the above-described features or techniques. The isolation circuit may provide intrinsic reverse voltage protection via transistor 310 (Q2) and sustained current via diode D1 and the combination of resistors 312 (R3) and 314 (R4). This may further reduce the physical area required for the isolation circuit by enabling the elimination of other circuit components. Note that, in some implementations, isolation may be expanded to additional paths (e.g., digital paths), by adding a switch to the path with a control signal connected to the gate of transistor 310 (Q2). The control signal may be, e.g., an N-type Field Effect Transistor (NFET) gate.

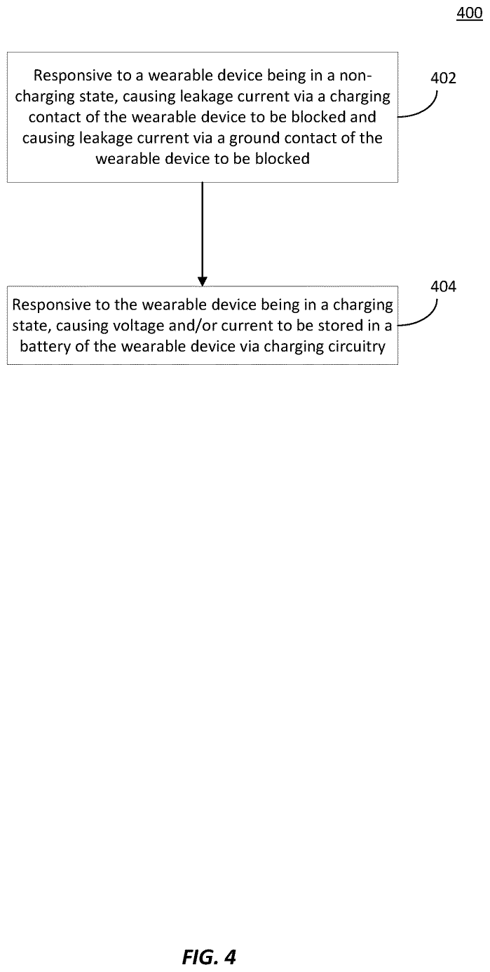

Turning to FIG. 4, a flowchart of an example process 400 for power and ground path isolation is provided in accordance with some embodiments. Blocks of process 400 may be performed by an isolation circuit, e.g., as shown in and described above in connection with FIGS. 2 and 3. In some embodiments, blocks of process 400 may be performed in an order other than what is shown in FIG. 4. In some implementations, two or more blocks of process 400 may be performed substantially in parallel. In some implementations, one or more blocks of process 400 may be omitted.

At 402, process 400 can, responsive to a wearable device being in a non-charging state, cause leakage current via a charging contact of the wearable device to be blocked, and cause leakage current via a ground contact of the wearable device to be blocked. In some implementations, leakage current via the charging contact may be blocked by a first transistor, which may be in an OFF state, as shown in and described above in connection with FIG. 3. In some implementations, leakage current via the ground contact may be blocked by a second transistor, which may be in an OFF state, as shown in and described above in connection with FIG. 3. Additionally or alternatively, in some embodiments, leakage current may be blocked via the ground contact by a diode.

At 404, responsive to the wearable device being in a charging state, process 400 can cause voltage and/or current to be stored in a battery of the wearable device via charging circuitry. For example, the isolation circuit may cause the first transistor (that blocked leakage current via the charging contact) to transition to an ON state, and may cause the second transistor (that blocked leakage current via the ground contact) to transition to an ON state. In some implementations, state transitions of the first transistor and/or the second transistor may be controlled by a third transistor, as shown in and described above in connection with FIG. 3. For example, the third transistor may transition to an ON state responsive to an external charger being operatively coupled to the wearable device. The third transistor may transition to the ON state with a delay controlled by a resistor and capacitor operatively coupled to the third transistor, as shown in FIG. 3. The first transistor and the second transistor may be operatively coupled to the third transistor such that the transition of the third transistor to the ON state causes the first transistor and the second transistor to transition to the ON state, as shown in and described above in connection with FIG. 3. The delay in transition to the ON state may be used (e.g., by a processor associated with the external charger) to detect liquid or foreign objects on the charging contacts of the wearable device.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

Specific details are given in the description to provide a thorough understanding of the embodiments. However, embodiments may be practiced without these specific details. For example, well-known circuits, processes, systems, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing various embodiments. Various changes may be made in the function and arrangement of elements without departing from the spirit and scope of the present disclosure.

Also, some embodiments were described as processes depicted as flow diagrams or block diagrams. Although each may describe the operations as a sequential process, many of the operations may be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It will be apparent to those skilled in the art that substantial variations may be made in accordance with specific requirements. For example, customized or special-purpose hardware might also be used, and/or particular elements might be implemented in hardware, software (including portable software, such as applets, etc.), or both. Further, connection to other computing devices such as network input/output devices may be employed.

With reference to the appended figures, components that can include memory can include non-transitory machine-readable media. The term “machine-readable medium” and “computer-readable medium” may refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including, but not limited to, non-volatile media, volatile media, and transmission media. Common forms of computer-readable media include, for example, magnetic and/or optical media such as compact disk (CD) or digital versatile disk (DVD), punch cards, paper tape, any other physical medium with patterns of holes, a RAM, a programmable read-only memory (PROM), an erasable programmable read-only memory (EPROM), a FLASH-EPROM, any other memory chip or cartridge, a carrier wave as described hereinafter, or any other medium from which a computer can read instructions and/or code. A computer program product may include code and/or machine-executable instructions that may represent a procedure, a function, a subprogram, a program, a routine, an application (App), a subroutine, a module, a software package, a class, or any combination of instructions, data structures, or program statements.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that are also expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, etc.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific embodiments have been described, these are not intended to be limiting. Various modifications and equivalents are within the scope of the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A method for power and ground path isolation, the method comprising:
responsive to a wearable device that includes a battery and a charging circuit being in a non-charging state:
causing current between a charging contact of the wearable device and the charging circuit to be blocked via a first electronic component and causing current between a ground contact of the wearable device and the charging circuit to be blocked via a second electronic component, wherein the charging contact and the ground contact are disposed on a portion of the wearable device configured to be in contact with skin of a wearer of the wearable device; and
responsive to the wearable device being in a charging state:
unblocking, via the first electronic component, the current between the charging circuit and the charging contact and unblocking, via the second electronic component, the current between the charging circuit and the ground contact, and
causing, via the charging circuit, electrical energy from an external charger operatively coupled to the wearable device to be stored in the battery of the wearable device.

13. The method of claim 12, wherein:
the first electronic component comprises a first transistor; and
causing current between the charging contact and the charging circuit to be blocked via the first electronic component comprises causing the first transistor to be in an off state.

14. The method of claim 13, wherein:
the second electronic component comprises a second transistor; and
causing current between the ground contact and the charging circuit to be blocked via the second electronic component comprises causing the second transistor to be in an off state.

15. The method of claim 14, wherein during at least a portion of the charging state, the first transistor associated with the charging contact and the second transistor associated with the ground contact are both in an on state.

16. The method of claim 15, wherein the first transistor and the second transistor are transitioned to the on state by causing a third transistor to be in an on state.

17. The method of claim 16, wherein the third transistor is transitioned to the on state responsive to the external charger being operatively coupled to the wearable device, and wherein the third transistor transitions to the on state with a delay after the external charger is operatively coupled to the wearable device.

18. The method of claim 12, wherein the wearable device comprises a plurality of electrodes configured to sense physiological signals of the wearer.

19. The method of claim 18, wherein the plurality of electrodes comprise a plurality of electromyography (EMG) electrodes.

20. The method of claim 12, wherein the wearable device further comprises a bias electrode configured to apply a bias voltage to the skin of the wearer.
